(19) United States
(12) Patent Application Publication
(10) Pub. No.: US 2011/0034944 A1
(43) Pub. Date: Feb. 10, 2011

(54) SURGICAL INSTRUMENT FOR FIXING, PREFERABLY IN A MINIMALLY INVASIVE ARTHROSCOPIC MANNER, A CARTILAGINOID TISSUE TO A BONE

(76) Inventor: Reinhold Ganz, Guemligen (CH)

Correspondence Address: Mark J Pandiscio;Pandiscio & Pandiscio, 470 Totten Pond Road, Waltham, MA, 02451, US

(21) Appl. No.: 12/734,157

(22) PCT Filed: Jul. 28, 2008
(86) PCT No.: PCT/IB2008/002038

§ 371 Date: Oct. 20, 2010

(30) Foreign Application Priority Data
Sep. 18, 2007 (IT) MI2007A001796

(51) Int. Cl. A61B 17/34

(52) U.S. Cl. 606/185

(57) ABSTRACT

A surgical instrument (5) for fixing, preferably in a minimally invasive arthroscopic manner, a cartilaginoid tissue to an underlying bone, in particular to an acetabular lip, accidentally detached from the bone edge of a femoral acetabulum is characterized in that said instrument comprises a proximal beating mass (2), which can be removably engaged with a proximal end (8) of a middle part (3) having the distal end (13) thereof designed for supporting a replaceable fixing device (21,22) which can be removably coupled to said distal end of said middle part, and a cannula part (5) designed for slidably receiving therein said middle part with said fixing device removably coupled thereto.

### BACKGROUND OF THE INVENTION

The present invention relates to surgical instrument for fixing, preferably in a minimally invasive arthroscopic manner, a cartilaginoid tissue to an underlying bone, with particular reference to an acetabular lip accidentally detached from the osseous edge or rim of a femoral acetabulum.

As is known, the fixing of an acetabular lip which, for several reasons, has been detached from an underlying bone, is at present carried out by suture anchoring elements, similar to those used in shoulder surgical operations, which frequently require a very invasive so-called “open sky” intervention.

In particular, for applying the above mentioned suture anchoring elements, it is necessary to properly prepare the hip bone, by forming at least a pre-hole and engaging in said pre-hole a dedicated fixation or clamping device.

Then, to the head of the above device one or more suture threads, for fixing the cartilaginoid tissue, for example, to the articular lip, are connected.

For each anchoring element, it is necessary to repeat the just disclosed operations, which greatly extends the surgical operation time.

Moreover, it is very difficult to perform, by the above method, an arthroscopic type of operation and, accordingly, the fixing operation must be carried out by using a substantially conventional type of surgical instrument or tool, with the risk of greatly damaging the underlying bone parts.

### SUMMARY OF THE INVENTION

Accordingly, the main aim of the present invention is to overcome the above mentioned drawbacks of prior fixing methods, by providing a novel fixing surgical instrument, specifically designed to perform a minimally invasive fixing operation, in particular an arthroscopic fixing operation.

Another object of the present invention is to greatly reduce the surgical operation time, while providing a novel surgical fixing or fixation instrument, adapted to perform a much more simple and quick surgical fixing operation.

Another object of the present invention is to provide such a novel surgical instrument providing a safe reliable fixing of the cartilaginoid tissue, and which is construction wise very simple and reliable, and which, moreover, allows to perform a consistently repeatable fixing intervention.

Another object of the present invention is to provide such a minimally invasive quickly operating surgical instrument allowing fixing operations to be performed arthroscopically, thereby greatly reducing possible risks related to the surgical operation, such as an infective, anaesthesia and blood loss risks.

Another object of the present invention is to provide such a surgical instrument capable of fixing the cartilaginoid tissue to the bone without drilling pre-holes or grooves in the bone.

Another object of the present invention is to provide such a surgical instrument allowing to fix the cartilaginoid lip without the need of performing fixing manual operations to clamp, by suture threads, the cartilaginoid tissue to the underlying bone.

Yet another object of the present invention is to provide such a surgical instrument which can be easily used in any operating rooms, requires a minimum maintenance and, moreover, is very competitive from a mere economic standpoint.

According to one aspect of the present invention, the above mentioned objects, as well as yet other objects, which will become more apparent hereinafter, are achieved by a surgical instrument for fixing, preferably in a minimally invasive arthroscopic manner, a cartilaginoid tissue to an acetabular lip accidentally detached from a. bone rim of a femoral acetabulum, characterized in that said instrument comprises a proximal beating mass, having a portion removably engageable with the proximal end of a middle portion having a distal end thereof adapted to support a replaceable fixing device which can be removably coupled to said distal end of said middle part, and a cannula part adapted to slidably receive therein said middle part with said fixing device removably coupled thereto.

### BRIEF DESCRIPTION OF THE DRAWINGS

Further characteristics and advantages of the surgical instrument according to the present invention will become more apparent hereinafter from the following detailed disclosure of a preferred, though not exclusive, embodiment thereof, which is illustrated, by way of an indicative, but not limitative example, in the accompanying drawings, where:

FIG. 1 is an exploded perspective view, showing the main component parts of the surgical instrument or tool according to the present invention;

FIG. 2 is a partial perspective view showing possible geometrical configurations of respective fixing elements which can be applied by the surgical instrument shown in FIG. 1;

FIG. 3 is a detail perspective view showing a possible configuration or embodiment of a cartridge and/or block supporting element, specifically designed for slidably supporting thereon the fixing element or device shown in FIG. 2;

FIGS. 4A (from a to c) and 4B (from a′ to c′) show further partial perspective view of parts of the surgical instrument according to the present invention, useful for understanding the operation of this instrument;

FIG. 5 is a further detail view, as partially cross-sectioned, showing a detail of the surgical instrument according to the present invention, with the fixing device already engaged in its cartridge and cannula and ready for fixing an acetabular lip to an underlying bone (herein not shown);

FIG. 6 is yet another perspective view illustrating an upper or top detachment of the acetabular lip;

FIG. 7 is yet another perspective view showing the surgical instrument according to the present invention in a ready condition to perform a fixing operation of the acetabular lip; and

FIG. 8 is yet another perspective view showing an operating mode of the surgical instrument according to the present invention, after having fixed the acetabular lip to the underlying bone, by applying a single fixing or clamping element or device.

### DESCRIPTION OF THE PREFERRED EMBODIMENTS

With reference to above mentioned figures, in FIG. 1 the surgical instrument or tool according to the present invention, has been generally indicated by the reference letter S.

Said instrument comprises a beating mass, generally indicated by the reference number 2, a middle part, generally indicated by the reference number 3, a fixing device bearing carriage, generally indicated by the reference number 4, and a cannula, generally indicated by the reference number 5.

The beating mass 2 of the surgical instrument S comprises, in turn, a substantially solid cylindric beating or impacting head 6, integral with a central stem, also of a substantially solid cylindric configuration, 7, having a diameter less than that of the beating or impacting head 6.

The middle part 3 of the surgical instrument S defines a substantially cylindric hollow proximal end portion 8, having at least a guide slotted longitudinal opening 9, for removably slidingly engaging therein the central stem 7 of the beating mass 2.

Said cylindric hollow portion 8 has an end face 10, operating as a closure bottom for the cylindric portion 8 and therefrom integrally extends a rod-like element, of substantially cylindric configuration 11, having a diameter much smaller than that of the cylindric hollow portion 8, and ending with a substantially flat distal end portion, generally indicated by the reference number 13.

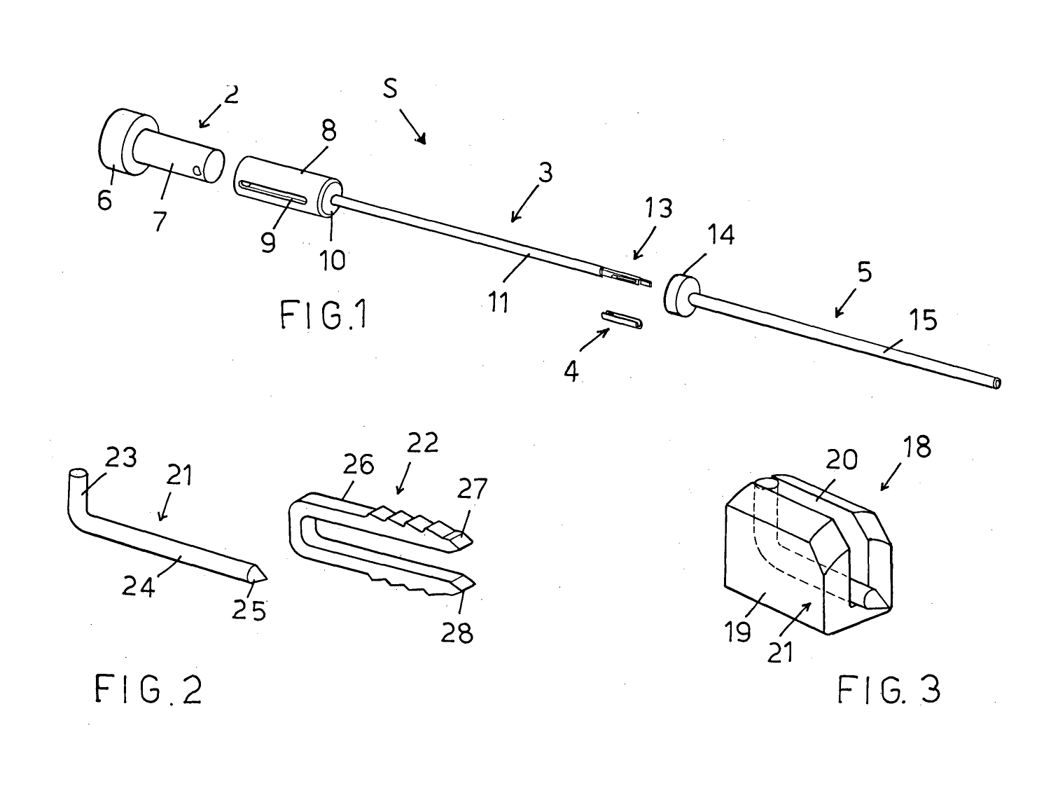

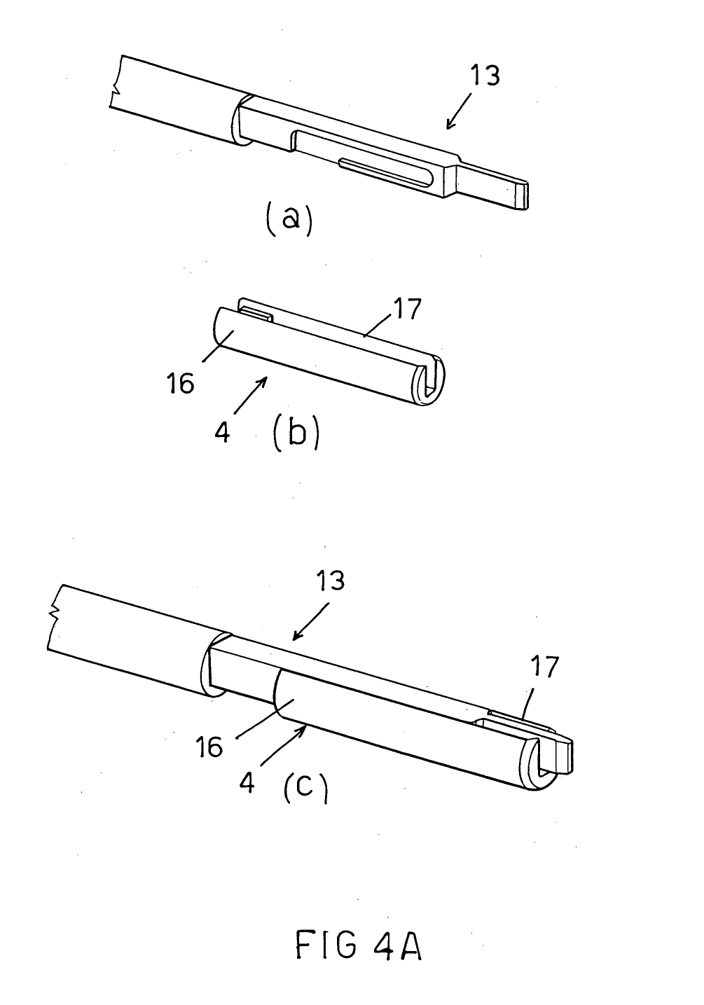

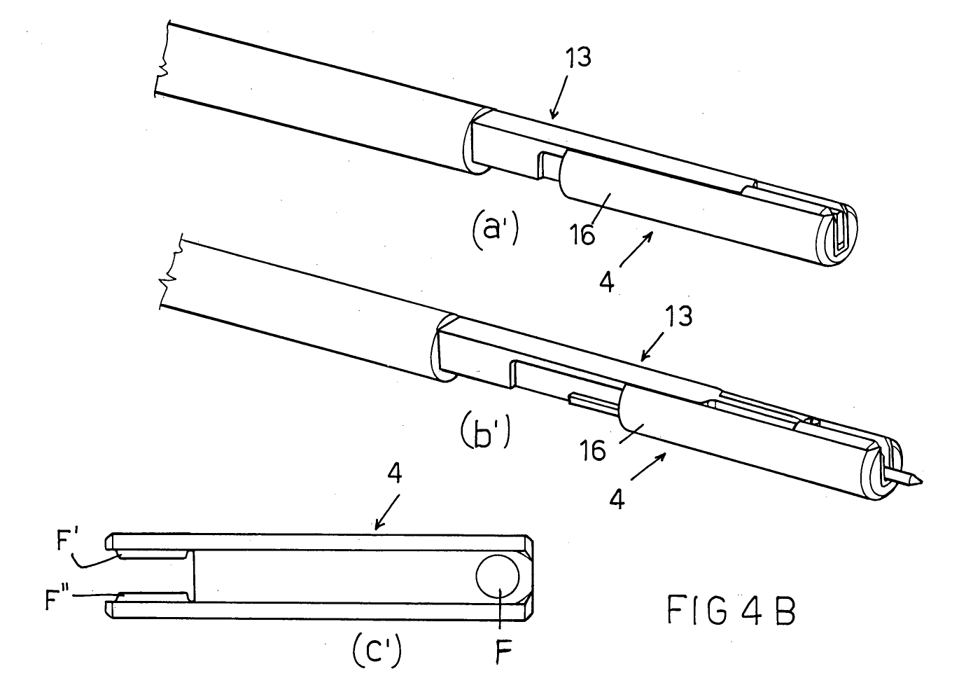

The cannula part 5, in turn, comprises a substantially cylindric hollow head portion 14, designed for abutting, with the rod 11 being engaged in the cannula, against the face 10 of the cylindric hollow portion 8 of the middle part 3, and a cylindric thin cannula portion 15, extending substantially centrally and integrally from the head portion 14 of the cannula 5, and having a diameter substantially less than that of said head portion 14. The carriage element 4, which is shown in a more detailed manner in the perspective view of FIG. 4 (b) and in the top plan view in FIG. 4B (c′), comprises, in turn, an elongated cylindric or plug body 16 having a substantially longitudinal central slot 17, for engaging therein a cartridge and/or block element, generally indicated by the reference number 18 in FIG. 3, having a block body 19 in which is formed a longitudinal slot 20 in which is slidably engaged a fixing device, generally indicated by the reference numbers 21 and 22 in FIG. 2, respectively.

More specifically, according to a first embodiment thereof, the fixing device 21 comprises a substantially L-shape hook element, having a short arm 23 and a long arm 24.

Advantageously, the long arm 24 ends with a sharpened pointed portion 25.

Said fixing devices 21 and 22 may have different lengths, sizes and cross sections, to perfectly fir specific requirements of the patient, in particular, for properly resisting against any withdrawal and twisting forces applied to the fixing device.

More specifically, the fixing device 21 has a substantially circular cross-sections, whereas the fixing device 22 comprises a clip element 26, of substantially U-shape, and having two respective end spaced tip portions 27 and 28, said clip element 26 further advantageously including surface teeth for improving its anchoring to the bone, after having applied by impacts said clip element 26 to the bone, through the surgical instrument S, as it will become more apparent hereinafter.

According to an important aspect of the present invention, the fixing device, which has substantially a L-hook, or a U-clip shape, comprises moreover, in addition to one or two sharpened tips, a surface textured portion, to prevent it from being accidentally withdrawn from the bone under accidental stresses.

Advantageously, the clamping device 21 or 22 is made of a biocompatible material, adapted to prevent or minimize possible troubles due, for example, to possible clinical analyses, such as NMR analyses, necessary to evaluate a good fixation of the cartilaginoid tissue to the bone and, accordingly, the recovery course of the patient.

Thus, the above mentioned biocompatible material must be of non magnetic nature, and capable to minimize any possible artefacts.

By way of an example, the fixing device can be made of titanium and alloys thereof, biocompatible polymers or other metals or metal alloys, either of a shape memory or of a standard type, provided that they have the above mentioned features.

Thus, the fixing devices 21 or 22, respectively the hook element, or U-shape element, can be engaged in the bone, as it will become more apparent hereinafter, without the need of preliminarily drilling a hole or a recess in said bone, thereby advantageously reducing the operating time and, accordingly, any risks of the surgical intervention (such as infections, anaesthesia or hematic losses).

In FIG. 3, the fixing device 21 has been shown as slidably supported in said support cartridge 18, which cartridge is advantageously of a disposable type and, in addition to operate for containing the fixing device therein, can be removably slidably engaged in said slot 17 of the body 16 of the carriage 4, to allow the device to be easily handled and engaged by the instrument S.

Actually, as stated, said cartridge and/or block 18 comprises a longitudinal slot 20 restraining therein the fixing device (23 in FIG. 3) and properly guiding said device as it is impacted into the bone, as it will become more apparent hereinafter.

With reference now to FIGS. 4A to 4B, the fixing device supporting carriage 4 can be engaged/assembled in/to the middle part 3, and, in particular, in/to its flat tip portion 13, through the longitudinal slot 17 of the carriage 4.

This coupling to, and a corresponding disengaging from said middle part 3, can be performed in a very easy manner, for example by a snap type of operation.

Actually, the carriage 4 can be easily disassembled or detached by removing the fixing device supporting cartridge 18 from its seat (FIG. 4A, c) (FIG. 4B, a′), thereby allowing the surgical instrument, and in particular, the middle part 3 thereof, to be easily cleaned and sterilized.

On the contrary, with the cartridge 18 arranged in its seat (FIG. 4B, b′), said carriage cannot be detached or disassembled, thereby allowing said fixing devices 21 and/or 22, which have a very small size, to be easily handled or driven, thereby preventing them from being accidentally disengaged (FIG. 4B, c′). Finally, as is shown in FIG. 4B, c′, the carriage 4 also comprises a hole F for allowing the cartridge 18 to be easily removed, as necessary, and suitable machined regions, such as locking ribs F′, F″, for preventing the cartridge from being ejected in a longitudinal direction.

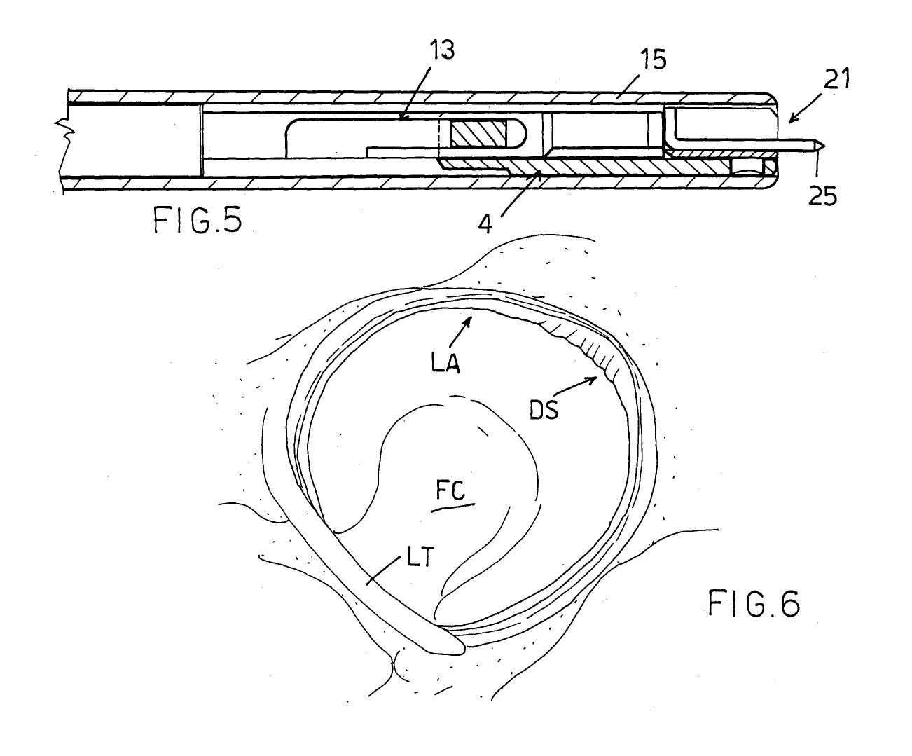

In this connection it should be pointed out that the cartridge 18 for supporting the fixing device 21 and/or 22 will be pre-assembled with the fixing device itself, thereby causing its tip portion 25, or 27 and/or 28, to project from the instrument S in an assembled condition of the latter. Thus, it is possible to easily grip the cartilaginoid tissue and fix this tissue at any desired positions, without using other tools or operators, at it is clearly shown in FIG. 5.

Thus, the cartridge 18 and inner portion of the surgical instrument S will prevent the device being impacted upon from deviating from a desired operating path. Actually, the instrument S is operatively impacted by causing the beating mass 2, made integral with the inner part 3, to slide in the guide groove and/or elongated slot 9 of the inner part of the instrument S.

Accordingly, as it will become apparent to one skilled in the art, the inventive surgical instrument S allows to implant any desired number of fixing devices 22 and/or 23, without removing said cannula 15, and this by merely withdrawing or removing the inner part 3 and the carriage 4 from the cannula 15, while also removing the empty cartridge 18 (which operation can be easily performed due to the provision of said hole F of the carriage 4), and by engaging a fresh cartridge and related fixing device 21 and/or 22.

FIG. 5 is a partial cross-section view illustrating the fixing device 21 supported in its supporting cartridge. 18, and engaged in the respective cannula 15.

FIG. 6 shows a schematic view of the acetabular lip LA, having an upper or top detached portion DS.

In particular, the cotyloid fossa FC, the articular hyalinic cartilage CIA, and the acetabular transverse ligament LT are herein shown.

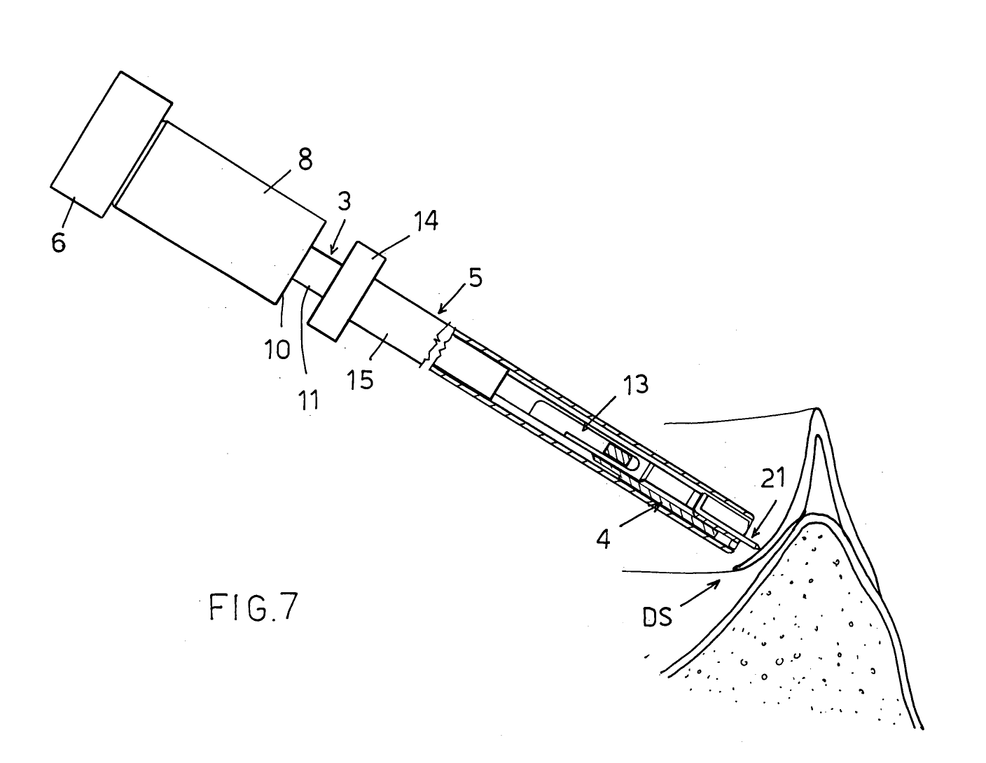

FIG. 7 is a schematic view provided for clearly understanding the operation of the inventive surgical instrument S.

In particular, in FIG. 7, the instrument tp 25 is shown arranged in the cannula, after having engaged the middle part of the instrument S, near the lip L to be fixed.

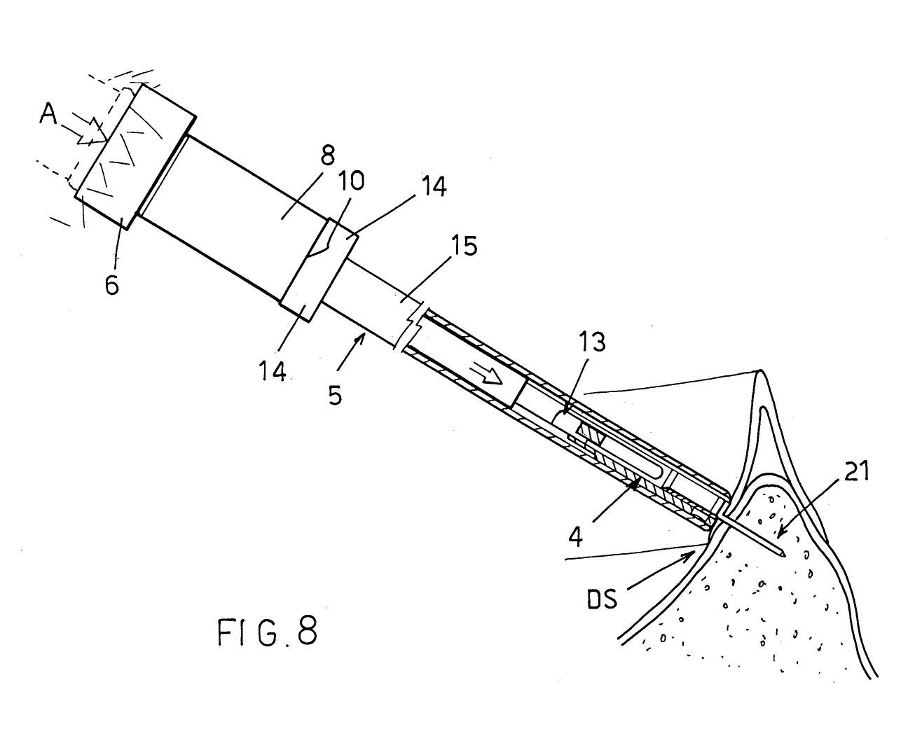

As shown in FIG. 8, by applying suitable repeated impacts to the head 6 of the beating mass 2 in the direction of the arrow A, for example by a mallet manually driven by the surgeon (not shown), the tip or point portion 5 will be caused to enter both the acetabular lip LA and the underlying bone, thereby completing the fixing operation, by causing the fixing device 21 to be deeply engaged in the bone.

In this connection it should be apparent that this operation can be easily and quickly repeated to provide a perfect fixation by further fixing hook elements 21 to be applied at any desired position, as chosen by the surgeon.

Thus, it is apparent that the inventive surgical instrument S allows to implant any desired number of fixing devices 21 and/or 22, without removing the implanted cannula, but merely withdrawing the inner part 3 and the carriage 4 from the cannula, while removing the empty cartridge 18 (which operation can be easily performed owing to the provision of the carriage hole F), and by introducing a fresh cartridge and related fixing device 21 and/or 22.

From the above disclosure it should be apparent that the invention fully achieves the intended aim and objects.

While the invention has been disclosed with reference to preferred embodiments, it should be apparent that the disclosed embodiments are susceptible to several modifications and variations, all of which will come within the scope of the invention.

In practicing the invention, the used contingent materials and/or sizes and/or shapes can be any, depending on requirements.

1. A surgical instrument for fixing, preferably in a minimally invasive arthroscopic manner, a cartilaginoid tissue to an acetabular lip accidentally detached from a bone rim of a femoral acetabulum, characterized in that said instrument comprises a proximal beating mass, having a portion removably engageable with the proximal end of a middle portion having a distal end thereof adapted to support a replaceable fixing device which can be removably coupled to said distal end of said middle part, and a cannula part adapted to slidably receive therein said middle part with said fixing device removably coupled thereto.

2. A surgical instrument according to claim 1, characterized in that said fixing device is substantially snap removably coupled to said distal end of said middle part by a carriage element removably supporting therein said fixing device.

3. A surgical instrument according to claim 1, characterized in that said fixing device comprises an elongated body having at least a pointed and sharpened end portion designed for perforating, as said beating mass is subjected to repeated impacts, said cartilaginoid tissue of said acetabular lip and said bone rim, to fix said acetabular lip to said bone rim, while penetrating said cartilaginoid tissue and bone rim.

4. A surgical instrument according to claim 3, characterized in that said elongated body of said fixing device has a substantially L-shape, said substantially L-shape defining L arms having a substantially cylindric cross-section.

5. A surgical instrument according to claim 3, characterized in that said elongated body of said fixing device has a substantially U-shape clip configuration, defining two pointed and sharpened portions, said U-shape having arms of a substantially rectangular and/or square cross-section.

6. A surgical instrument according to claim 1, characterized in that said fixing device is slidably engaged in an engaging slot of a disposable cartridge and/or block element, said disposable cartridge and/or block element being in turn removably slidably engaged in a corresponding longitudinal slot of a carriage body, said at least a pointed portion of said fixing body projecting, in an assembled and inoperative condition of said instrument, from a surface of said disposable cartridge and/or block element.

7. A surgical instrument according claim 1, characterized in that said proximal beating mass comprises a substantially cylindric solid impact head integral with a central stem, also having a substantially cylindric solid configuration and a diameter much less than a diameter of said impact head.

8. A surgical instrument according to claim 1, characterized in that said proximal end of said middle part of said surgical instrument is a substantially cylindric hollow portion having at least a longitudinal guide slot to be removably coupled to said central stem of said proximal beating mass, said cylindric hollow portion having a bottom end face therefrom integrally extends a substantially cylindric rod element, having a diameter much less than the diameter of said cylindric hollow portion and ending in said distal end of said middle portion, said distal end being of essentially flat configuration.

9. A surgical instrument according to claim 1, characterized in that said cannula part has a substantially cylindric hollow head portion adapted to abut, with said instrument in an assembled condition, against said end surface of said cylindric hollow portion of said middle part, and a cannula portion extending substantially centrally integrally from said head portion of said cannular part.

10. A surgical instrument according to claim 9, characterized in that said cannula part is so designed as to be permanently retained in an implanted condition during a fixation operation, while allowing said fixation operation to be arthroscopically performed in a non-open sky condition.

11. A surgical instrument according to claim 1, characterized in that said carriage element is snap engaged with said flat distal end of said middle part, thereby allowing said carriage element to be easily and quickly disassembled as said fixing device supporting cartridge is removed from said carriage element, thereby allowing said surgical instrument to be easily cleaned and sterilized.

12. A surgical instrument according to claim 11, characterized in that said carriage element comprises at least a perforation to facilitate a removal of said fixing device supporting cartridge, said carriage element being so machined as to prevent said cartridge from being ejected in a longitudinal direction.

13. A surgical instrument according to claim 1, characterized in that said middle part is so contoured as to be coupled to said carriage element to allow said fixing device to be impacted in a guided manner.

14. A surgical instrument according to claim 1 and claim 13, characterized in that said fixing device is implanted by impacts by causing said beating mass, made integral with said inner part, to slide in said guide slot formed in said inner part of said surgical instrument.

15. A surgical instrument according to claim 1, characterized in that said fixing device, having a L or U shaped clip and said sharpened tip or point portion, comprises moreover a macro-textured and/or surface processed surface to prevent said fixing device from being accidentally withdrawn from a respective bone said fixing device is applied to.

16. A surgical instrument according to claim 15, characterized in that said fixing device is made of a biocompatible material to prevent or minimize troubles in performing conventional clinic analyses, such as NMR, to evaluate a proper fixation of the cartilaginoid tissue to the bone.

17. A surgical instrument according to claim 16, characterized in that said material of said fixing device is a non magnetic material for minimizing artifacts, said material comprising titanium and alloys thereof, biocompatible polymers and/or metals or metal alloys either of a shape memory type or a standard type.

18. A surgical instrument according to claim 1, characterized in that said fixing device is engaged in said bone without preliminarily perforating and/or grooving said bone.

19. A surgical instrument according to claim 1, characterized in that said fixing device is so designed as to be implanted upon having gripped said cartilaginoid tissue through said pointed and sharpened end of said fixing device, thereby arranging said cartilaginoid tissue between said bone and the other end portion of said device.

20. A surgical instrument for fixing, preferably in a minimally invasive arthroscopic manner, a cartilaginoid tissue to an underlying bone, according to one or more of the preceding claims and substantially as broadly disclosed and illustrated for the intended objects.
